(19) United States
(12) Patent Application Publication
(10) Pub. No.: US 2024/0271922 A1
(43) Pub. Date: Aug. 15, 2024

(54) OPTICAL MEASUREMENT APPARATUS AND METHOD OF MEASURING AN AXIAL LENGTH

(71) Applicant: Occuity Limited, London (GB)

(72) Inventors: James REYNOLDS, Gloucestershire (GB); Robin TAYLOR, Berkshire (GB)

(73) Assignee: Occuity Limited, London (GB)

(21) Appl. No.: 18/563,801

(22) PCT Filed: May 16, 2022
(86) PCT No.: PCT/EP2022/063180

§ 371 Date: Nov. 22, 2023

(30) Foreign Application Priority Data
May 26, 2021 (GB) 2107472.9

(51) Int. Cl. G01B 9/02015; G01B 9/02; G01B 9/0209; G01B 11/14

(52) U.S. Cl. CPC G01B 9/0203; G01B 9/02042; G01B 9/0209; G01B 11/14

(57) ABSTRACT

An optical measurement apparatus (100) combines confocal measurement and low coherence interferometric measurement. The apparatus (100) comprises an interferometric measurement subsystem (102) and a confocal measurement subsystem (104) disposed within a housing (101). An optical combiner (136) is configured to provide the interferometric measurement subsystem (102) and the confocal measurement subsystem (104) with irradiative access to a region to be measured located at a substantially static target location. The interferometric measurement subsystem (102) and the confocal measurement subsystem (104) are respectively configured to image longitudinally in respect of a first subregion (162) and a second subregion (164) in the region to be measured. the second subregion (164) being axially spaced from the first subregion (162). A processing resource (152. 154) is operably coupled to the interferometric measurement subsystem (102) and the confocal measurement subsystem (104), and configured to calculate a first range in respect of the first subregion (162) and a second range in respect of the second subregion (164).

The present invention relates to an optical measurement apparatus of the type that, for example, measures distances in respect of subregions in a region to be measured located at a substantially static target location. The present invention also relates to a method of measuring axial length, the method being of the type that, for example, measures a distance in a region to be measured located at a substantially static target location.

In the field of metrology, measurement of a separation between a retina of an eye and an anterior surface of a cornea is useful as a primary determinant for the diagnosis of myopia, i.e. a patient with myopia typically has a relatively large axial length between the retina and the anterior surface of the cornea. Excessive axial length of this kind is partially a result of a patient's genetics and partially environmental causes, and generally occurs as the eye grows during childhood. A number of corrective mechanisms exist to decrease eye growth. However, successful use of these mechanisms requires the axial length of the eye of the patient to be monitored. With the prevalence of myopia, a simple low-cost instrument, capable of noncontact measurement, and which is handheld, is desirable.

To measure the axial length of the eye, it is necessary to determine the distance to both the retina and the apex of the cornea simultaneously. When optical means are used to make measurements without contacting the eye, simultaneous measurement is necessary in order to avoid errors attributable to movement of the eye during measurement.

It is understood that a particularly practicable optical method of measuring the distance to the retina of the eye is Low Coherence Interferometry (LCI). In principle, it is possible to use a single LCI measurement system to measure both the distance to the retina and the distance to the cornea simultaneously. However, in practice where a time domain LCI measurement system is employed, the distances to the retina and cornea are relatively widely separated and a significant amount of time will elapse during translation of a scanning system carrying an objective lens between two linear distances corresponding to measurement of the cornea and the retina. In this regard, simultaneous measurement is not possible owing to the possibility of the eye moving involuntarily during elapse of the translation time.

As an alternative to using the time domain LCI measurement system, a Fourier domain variant of the LCI measurement system provides intrinsically substantially simultaneous measurement. However, the maximum measurement range of the Fourier domain LCI measurement system is much smaller than the separation of the retina and cornea in the eye.

Furthermore, in order to obtain an optical return signal from the retina that is sufficiently strong to enable peak detection, an emitted optical measurement beam needs to be approximately collimated, because the cornea and lens of the eye focuses the optical measurement beam into the region of the retina. However, as the cornea is convex in shape, little light from the collimated measurement beam is reflected back from the cornea into the time domain LCI measurement system. Therefore, in order to obtain a sufficiently strong return signal from the cornea, a second measurement beam, focussed into the region of the cornea, is required. Consequently, known practicable instruments that measure axial length use separate measurement beams for the cornea and the retina, and measure the axial distances to both using LCI.

Various instruments capable of measuring axial length are known, but are bulky, bench-mounted, instruments. As mentioned above, such instruments use LCI to measure both the axial position of the retina and the axial position of the cornea. For example, US patent publication no. 2007/076217 describes the use of two distinct LCI measurement systems to measure the axial positions of the cornea and retina, respectively. In contrast, US patent publication no. 2009/268209 describes a common LCI measurement system employing two reference beams of different path length and two emitted measurement beams addressing distinct and spatially separated surfaces.

Another known measurement technique, for example as described in Japanese patent publication no. 2-295533, employs a Mach-Zehnder/Fizeau type interferometer to compensate for different distances to the cornea and the retina using the difference between the paths in the Mach-Zehnder section of the interferometer. Similarly, as described in Japanese patent publication no. 2005-342204, a Michelson/Fizeau type interferometer is employed to compensate for different distances to the cornea and the retina using arm length differences in the Michelson section of the interferometer.

According to a first aspect of the present invention, there is provided an optical measurement apparatus combining confocal measurement and low-coherence interferometric measurement, the apparatus comprising: a housing; a confocal measurement subsystem disposed within the housing; an interferometric measurement subsystem disposed within the housing; an optical combiner configured to provide the confocal measurement subsystem and the interferometric measurement subsystem with irradiative access to a region to be measured located at a substantially static target location external to the housing; and a processing resource operably coupled to the confocal measurement subsystem and the interferometric measurement subsystem; wherein the confocal measurement subsystem is configured to image longitudinally in respect of a first subregion in the region to be measured; the interferometric measurement subsystem is configured to image longitudinally in respect of a second subregion in the region to be measured, the second subregion being axially spaced from the first subregion; and the processing resource is configured to calculate a first range in respect of the first subregion and a second range in respect of the second subregion.

The first subregion may be in respect of a first reflective interface. The second subregion may be in respect of a second reflective interface.

The apparatus may further comprise: an optical path internal to the housing; the optical path may extend from the optical combiner towards the region to be measured; and the internal optical path may be common to the confocal measurement subsystem and the interferometric measurement subsystem; and a length of the internal optical path may be fixed.

The processing resource may be configured to calculate, when in use, a confocal measurement and an interferometric measurement substantially contemporaneously over a measurement cycle.

The interferometric measurement subsystem may be substantially optically uninfluenced, when in use, by operation of the confocal measurement.

The confocal measurement subsystem may comprise at least one longitudinal imaging component; the interferometric measurement subsystem may comprise a measurement arm and a reference arm; and neither the measurement arm nor the reference arm of the interferometric measurement subsystem may comprise the at least one longitudinal imaging component.

The length of the internal optical path may be unchanged over the measurement cycle.

The confocal measurement subsystem may be operationally independent of the interferometric measurement subsystem.

The confocal measurement subsystem may be operably coupled to the interferometric measurement subsystem.

The apparatus may further comprise: projection optics disposed opposite a first side of the optical combiner; wherein the interferometric measurement subsystem may comprise a measurement arm configured to collimate, when in use, light propagating therethrough; an end of the measurement arm may be disposed opposite a second side of the optical combiner.

The measurement arm may comprise a collimating property.

The interferometric measurement subsystem may comprise: a reference arm arrangement comprising a plurality of different selectable optical path lengths; and a selection unit configured to select an optical path length of the plurality of selectable optical path lengths.

The reference arm may comprise a plurality of reference optical reflector elements. The selection unit may be operably coupled to the plurality of reference optical reflector elements.

15 The reference arm may comprise a plurality of different selectable optical fibres. The selection unit may be configured to select an optical fibre from the plurality of different selectable optical fibres.

The plurality of different selectable optical fibres may be of different length. A common reference optical reflector element may be disposed opposite respective free ends of the plurality of different selectable optical fibres.

The plurality of different optical fibres may be of a common length and a plurality of different reference optical reflector elements may be disposed 25 respectively at ends of the plurality of different optical fibres; the optical distances of each reference optical reflector element of the plurality of difference reference optical reflector elements from the respective ends of the plurality of different optical fibres may be the different.

30 The plurality of different selectable optical path lengths may comprise an optical fibre comprising a plurality of selectively reflective reflecting elements longitudinally spaced along the optical fibre. The plurality of selectively reflective reflecting elements may be operably coupled to the selection unit.

The plurality of different selectable optical path lengths may comprise an optical fibre comprising a fibre collimator disposed at an end thereof; the fibre collimator may be axially translatable and disposed opposite a reference optical reflector element. The fibre collimator may be coaxial with the reference optical reflector element. The selection unit may be configured to select an axial position of the fibre collimator.

The plurality of different selectable optical path lengths may comprise an optical fibre comprising a fibre collimator disposed at an end thereof; and a lens may be disposed between the fibre collimator and a plurality of partially reflective reference axially spaced optical reflector elements. Each partially reflective reference optical reflector element of the plurality of partially reflective reference optical reflector elements may be disposed coaxially with the fibre collimator and the lens. The selection unit may be configured to select an axial position of the lens.

The plurality of different selectable optical path lengths may comprise an optical fibre and a plurality of reflecting elements; each of the plurality of reflecting elements may be selectively disposable opposite an end of the optical fibre.

The confocal measurement subsystem may comprise a first translatable optical element and the interferometric measurement subsystem may comprise a second translatable optical element; the first and second optical elements may be configured to be translated substantially contemporaneously.

The first and second optical elements may be carried by a common translatable assembly.

The common translation assembly may have a translation range of between 3 mm and 15 mm.

The first optical element may be carried by a first translatable assembly and the second optical element may be carried by a second translatable assembly. The first translatable assembly may have a translation range of between 3 mm and 15 mm. The second translatable assembly may have a translation range of between 3 mm and 8 mm.

The first subregion may correspond to a cornea of an eye. The second subrange may correspond to a retina of an eye.

In accordance with a second aspect of the present invention, there is provided a method of measuring an axial length in a region to be measured located at a substantially static target location, the method comprising: longitudinally imaging using a confocal measurement subsystem of an optical measurement apparatus in respect of a first subregion in the region to be measured in order to make a first measurement; substantially contemporaneously longitudinally imaging using an interferometric measurement subsystem of the optical measurement apparatus in respect of a second subregion in the region to be measured in order to make a second measurement, the second subregion being axially spaced from the first subregion; and calculating a first range in respect of the first subregion and a range in respect of the second subregion.

The first subregion may be in respect of a first reflective interface. The second subregion may be in respect of a second reflective interface.

The method may further comprise: providing a calibration target; and calculating an offset between a first test measurement made by the confocal measurement subsystem in respect of the calibration target and a second test measurement made by the interferometric measurement subsystem in respect of the calibration target.

The method may further comprise: determining an effective refractive index of the region to be measured; and using the effective refractive index to make the first and second measurements.

The region to be measured may comprise a plurality of different contiguous subregions; the plurality of different contiguous subregions may comprise a plurality of different refractive indices, respectively. The method may comprise calculating a plurality of distances between the first and second subregions using the plurality of different refractive indices; and the plurality of distances may be summed, thereby providing the distance between the first and second subregions.

The optical measurement apparatus emits a substantially collimated beam of light in respect of the interferometric measurement subsystem.

The interferometric measurement subsystem may comprise a source of electromagnetic radiation configured to emit, when in use, light in a near-infrared region of the electromagnetic spectrum. A wavelength of the light emitted by the source of electromagnetic radiation may be less than 900 nm.

It is thus possible to provide an optical measurement apparatus and a method of measuring axial length that lends itself well to compact construction and thus portability in a hand-held form factor. The apparatus and method permit substantially contemporaneous measurement of axial distances to two reflective interfaces and thus an axial length between the two interfaces. Additionally, multiple implementations of the same measurement subsystem employing the same measurement technique are not required and substantially contemporaneous measurement of two different subregions of a sample can be performed, thereby obviating or at least mitigating measurement errors that can be encountered owing to movement of the sample.

At least one embodiment of the invention will now be described, by way of example only, with reference to the accompanying drawings, in which:

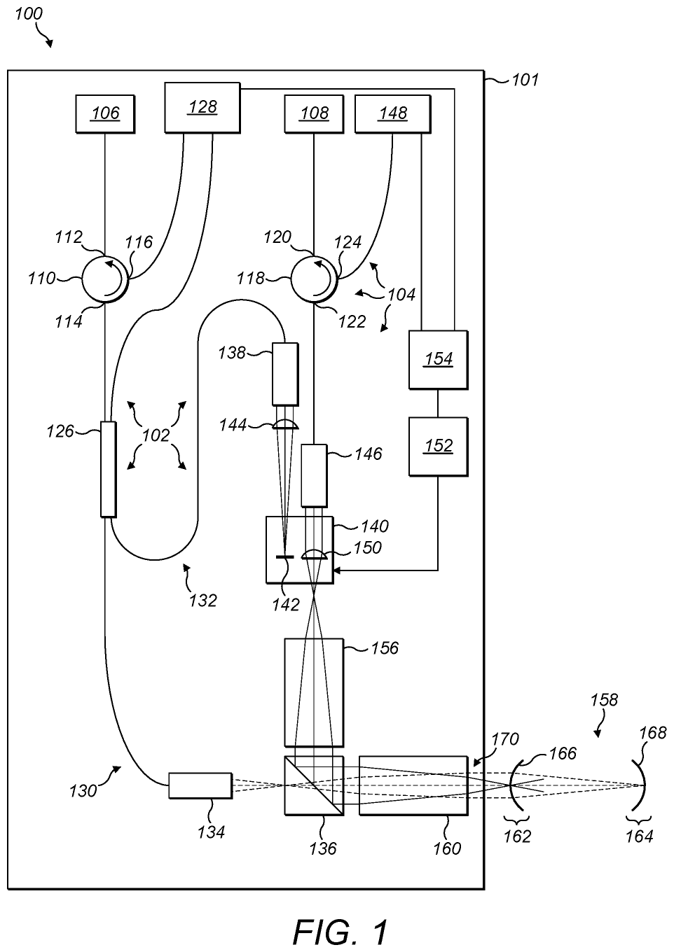

FIG. 1 is schematic diagram of an optical measurement apparatus constituting an embodiment of the invention;

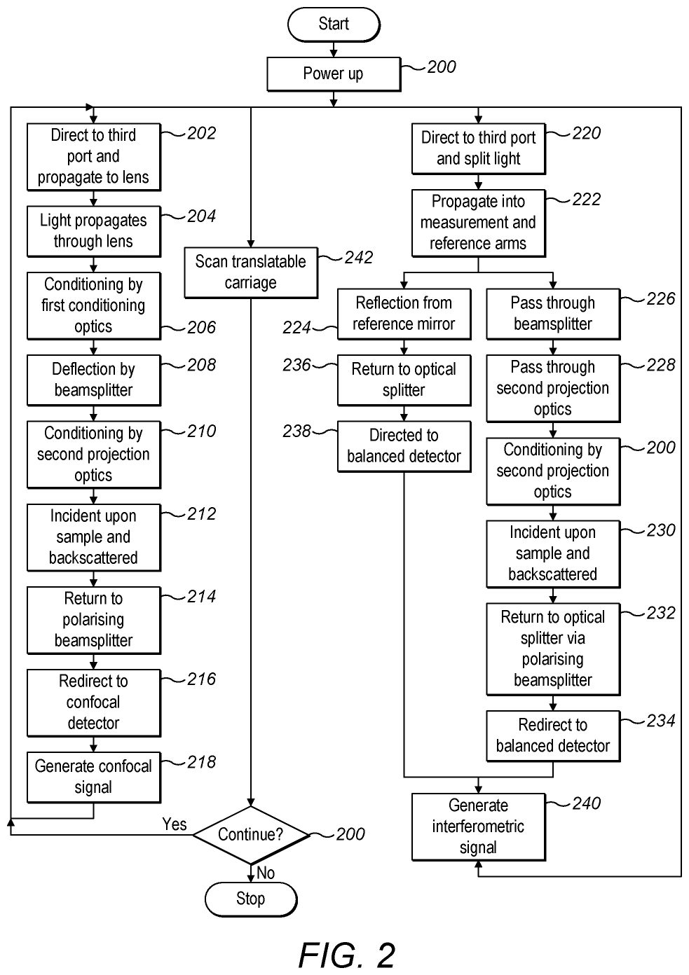

FIG. 2 is a flow diagram of a method of method of measuring an axial length; and

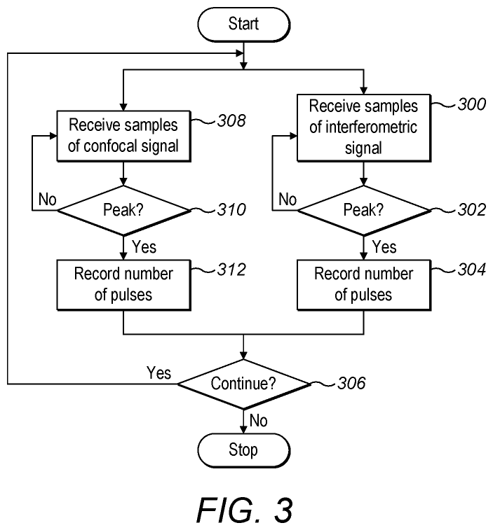

FIG. 3 is a flow diagram of determining ranges to subregions of a sample employed by the method of FIG. 2.

Throughout the following description identical reference numerals will be used to identify like parts.

Referring to FIG. 1, an optical measurement apparatus 100 combines a low-coherence interferometric measurement subsystem 102 with a confocal measurement subsystem 104. The apparatus 100 comprises, in this example, a housing 101 containing the low-coherence interferometric measurement subsystem 102 and the confocal measurement subsystem 104. The low-coherence interferometric measurement subsystem 102 comprises a first source of, at least partially coherent, electromagnetic radiation, for example a Superluminescent Light Emitting Diode (SLED) 106. To emit at least partially coherent electromagnetic radiation, the source of electromagnetic radiation 106 has a continuous spectrum and a bandwidth between about 1% and about 10% of a centre wavelength of the electromagnetic radiation emitted. The confocal measurement subsystem 104 comprises a second source of electromagnetic radiation, for example a laser diode 108. However, in other implementations, a common source of electromagnetic radiation can be employed, for example a common SLED, operably coupled to an optical splitter in order to provide sources of electromagnetic radiation to both the low-coherence interferometric measurement subsystem 102 and the confocal measurement subsystem 104.

In this example, the interferometric measurement subsystem 102 of the apparatus 100 comprises a first optical circulator 110 comprising a first port 112, a second port 114 and a third port 116. The first optical circulator 110 permits electromagnetic radiation incident at the first port 112 to pass through to the second port 114 and electromagnetic radiation incident at the second port 114 to pass through to the third port 116. Similarly, the confocal measurement subsystem 104 of the apparatus 100 comprises a second optical circulator 118 comprising a first port 120, a second port 122 and a third port 124. The second optical circulator 118 permits electromagnetic radiation incident at the first port 120 to pass through to the second port 122 and electromagnetic radiation incident at the second port 122 to pass through to the third port 124.

An output of the SLED 106 is operably coupled to the first port 112 of the first optical circulator 110 by optical fibre, the second port 114 of the first optical circulator 110 being operably coupled to a first port of an optical splitter 126 by optical fibre. The third port of the first optical circulator 110 is operably coupled to a first port of a balanced detector unit 128 by optical fibre. The balanced detector unit 128 can be any suitable arrangement of optical and optoelectronic devices, for example as described in “Balanced detection technique to measure small changes in transmission” (Houser et al., Applied Optics, 33 1059-1062 (1994)), and as the exact implementation of the balanced detector unit 128 is not core to an understanding of the operation of the embodiments described herein, the balanced detector unit 128 will not be described in any further detail. However, the skilled person should appreciate that other kinds of detector can be employed instead of the balanced detector unit 128, for example a photodiode detector or an avalanche photodiode (APD).

A second port of the balanced detector unit 128 is operably coupled to a second port of the optical splitter 126 by optical fibre. A third port of the optical splitter 126 is operably coupled to a measurement arm 130 of the low-coherence interferometric measurement subsystem 102, and a fourth port of the optical splitter 126 is operably coupled to a reference arm or branch 132 of the low-coherence interferometric measurement subsystem 102.

The third port of the optical splitter 126 is operably coupled to a focussing element 134, for example a lens, by optical fibre, the focussing element 134 being disposed opposite a first port of a polarising beamsplitter 136 constituting an optical combiner. The measurement arm 130 therefore comprises a collimating property. The optical fibre extending form the optical splitter 126 to the focussing element 134 and the focussing element 134 constitute, in this example the measurement arm 130 of the low-coherence interferometric measurement subsystem 102. The fourth port of the optical splitter 126 is operably coupled to a first fibre collimator 138 by optical fibre, the first fibre collimator 138 being disposed opposite a linearly translatable carriage 140 at the first side thereof. The first fibre collimator 138 is aligned with a reference mirror 142 carried by the translatable carriage 140. A first optical element, for example a focussing optical element, such as a first lens 144, is disposed between the first fibre collimator 138 and the translatable reference mirror 142. In another example, the first lens 144 can be disposed on the translatable carriage 140 so as to translate with the translatable carriage 140. In the present example, the optical fibre coupling the fourth port of the optical splitter 126 to the first fibre collimator 138, the first fibre collimator 138, the lens 144 and the reference mirror 142 constitute the reference arm 130 of the low-coherence interferometric measurement subsystem 102. The skilled person should appreciate though that other implementations are conceivable, for example the first fibre collimator 138 and the lens 144 can be incorporated into the optical fibre coupling the fourth port of the fibre splitter to the first fibre collimator 138 by way of providing a lensed fibre.

An output of the laser diode 108 is operably coupled to the first port 120 of the second optical circulator 118 by optical fibre. The second port 122 of the second optical circulator 118 is operably coupled to a second fibre collimator 146 by optical fibre, and the third port 124 of the second optical circulator 118 is operably coupled to a confocal detector unit 148 by optical fibre. The confocal detector unit 148 can be any suitable arrangement of optical and optoelectronic devices, for example a fibre-optic implementation, employing a pigtailed photodiode, of the arrangement described in UK patent no. 2 508 368. As the exact implementation of the confocal detector unit 148 is not core to an understanding of the operation of the embodiments described herein, the confocal detector unit 148 will not be described in any further detail.

The second port 122 of the second optical circulator 118 is therefore guided to a second optical element, for example as a second lens 150, mounted on the linearly translatable carriage 140. In this regard, the optical fibre with the optional second fibre collimator 146 is disposed opposite a first side of the linearly translatable carriage 140, which carries both the second lens 150 and the reference mirror 142 and so constitutes a common translatable assembly. In this example, the common translatable assembly has a translation range between about 3 mm and about 15 mm, for example about 10 mm. Both the reference mirror 142 and the second lens 150 are configured to be translated contemporaneously when in use. However, in other examples, separate translation mechanisms, for example translatable carriages, can be provided in order to translate the reference mirror 142 and the second lens 150 independently, but substantially contemporaneously. In such an embodiment, a first translatable assembly carrying the reference mirror 142 has a translation range of between about 3 mm and about 15 mm, and a second translatable assembly carrying the second lens 150 has a translation range of between about 3 mm and about 8 mm. In such an example, a controller can be provided to ensure synchromism between the first and second translatable carriages.

A processing resource comprises, for example a translation controller 152, such as a microcontroller, is operably coupled to the translatable carriage 140. The translatable carriage 140 carries an encoder scale (not shown), and a linear encoder (not shown) is disposed opposite the encoder scale and operably coupled to the translation controller 152. The combination of the linear encoder and the encoder scale is, for example, of the type described in UK patent no. GB 2 467 340, and serves to provide position feedback, when in use, with respect to the translatable carriage 140. Of course, if more than one translatable carriage is employed, then a corresponding greater number of encoder scales and linear encoders can be employed to obtain position feedback in respect of each translatable carriage. The processing resource 152 also supports a measurement unit 154, and is operably coupled to the translation controller 152 as well as the balanced detector unit 128 of the low-coherence interferometric measurement subsystem 102 and the confocal detector unit 148 of the confocal measurement subsystem 104.

The second lens 150 carried by the translatable carriage 140 is disposed opposite a first port of a first projection optics unit 156, which is disposed at a second side of the translatable carriage 140. A second port of the first projection optics unit 156 is disposed opposite a second port of the beamsplitter 136. The beamsplitter 136 serves to provide the interferometric measurement subsystem 102 and the confocal measurement subsystem 104 with irradiative access to a sample 158 described in further detail later below. Furthermore, the use of the beamsplitter 136 optimises the return of light reflected by the sample 158 to the low-coherence interferometric measurement subsystem 102 and the confocal measurement subsystem 104, but particularly the confocal measurement subsystem 104.

In this example, the optical combiner is a polarising beamsplitter, but in another embodiment the beamsplitter can be non-polarising and configured to provide an unequal power splitting ratio in order to return, for example, more light towards the confocal measurement subsystem 104 than the interferometric measurement subsystem 102, more specifically more light to the parts of the confocal measurement subsystem 104 upstream of the optical combiner 136 towards the second optical circulator 122 than the parts of the low-coherence interferometric measurement subsystem 102 upstream of the optical combiner 136 towards the first optical circulator 110. Of course, in other examples, the bias of the power splitting ratio can be in favour of the interferometric measurement subsystem 102.

A second projection optics unit 160, constituting a common sub-arm 132, is operably coupled to the beamsplitter 136 and provides an internal optical path for both the measurement arm 130 and the first projection optics unit 156, i.e. the internal optical path is common to the interferometric measurement subsystem 102 and the confocal measurement subsystem 104, thereby enabling the confocal measurement subsystem 102 operably coupled to the low-coherence interferometric measurement subsystem 104 to address the same region to be measured, but different subregions within the region to be measured. Furthermore, the internal optical path is internal to a housing 101 and extends from the beamsplitter 136 towards the sample 158. In this example, the length of the internal optical path is fixed and cannot be adjusted or configured by either of the measurement subsystems 102, 104. Indeed, the length of the internal optical path can be physically fixed. However, in other examples, the length of the internal optical path can be adjustable in length. A port 170 is provided for light to exit and re-enter the apparatus 100.

The sample 158, for example biological tissue, is in this example in vivo, for example an eye, such as a human eye, and is disposed opposite the second projection optics unit 160 and lies within the region to be measured. The reference mirror 142 is, in this example, disposed opposite the reference arm 132 and can linearly translate so as to move closer to and farther away from the reference arm 132. The sample 158 is, in this example substantially static during a measurement cycle, for example the apparatus 100 is not provided with a mechanism to move the sample 158 relative to the housing 101 and hence the second projection optics unit 160. Indeed, in this example, the internal optical path is unchanged over the measurement cycle. The shared use of the internal optical path, configured as set forth above, supports the provision of separate interferometric and confocal channels that can share the common sub-arm mentioned above substantially contemporaneously and without the confocal channel influencing or perturbing the interferometric channel and vice versa. In this regard, both the low-coherence interferometric measurement subsystem 102 and the confocal measurement subsystem 104 address the same target, for example the sample 158, at the same time.

In this example, the region to be measured comprises a first subregion 162 and a second subregion 164, the second subregion164 being axially spaced from the first subregion 162. In the context of an eye, the first subregion 162 corresponds to a cornea 166 and the second subregion corresponds to a retina 168. The first subregion 162, whether in the context of an eye or not, therefore comprises a first reflective interface and the second subregion 164 comprises a second reflective interface.

As will be apparent to the person skilled in the art, the first optical circulator 110, the SLED 106, the balanced detector unit 128, the optical splitter 126, the measurement arm 130, the reference arm 132, the reference mirror 142, the focussing element 134 and the first fibre collimator 138 constitute the low-coherence interferometric measurement subsystem 102. Similarly, the second optical circulator 112, the confocal detector unit 148, the laser diode 108, the second fibre collimator 146, the second lens 150 and the first projection optics unit 156 constitute the confocal measurement subsystem 104.

In operation (FIG. 2), the SLED 106 and the laser diode 108 are both powered up, as is the translatable carriage assembly and the processing resource (Step 200), and first electromagnetic radiation, for example coherent first electromagnetic radiation (hereafter referred to as “first light”) is emitted by the laser diode 108, although it is optional for the first electromagnetic radiation to be coherent, and second electromagnetic radiation, for example at least partially coherent second electromagnetic radiation (hereafter referred to as “second light”) is emitted by the SLED 106.

Referring to the confocal measurement subsystem 104, the first light emitted by the laser diode 108 propagates to the second optical circulator 118 and is directed (Step 202) by the second optical circulator 118 to the second port 122 thereof before propagating to the second fibre collimator 146 where the first light is collimated. The collimated first light is incident upon the second lens 150 (Step 202), which focusses (Step 204) the light to a point prior to entering the first projection optics unit 156. The focussed first light propagates through the first projection optics unit 156 and is conditioned (Step 206) by the optical elements contained therein before exiting the first projection optics unit 156 as approximately collimated first light. The first light is then incident upon the beamsplitter 136 where the first light is redirected (Step 208) by the beamsplitter 136 to the second projection optics unit 160.

The first light then propagates through the second projection optics unit 160 and is conditioned (Step 210) by the optical elements contained therein and exits the apparatus 100 via the port 170 and is focussed (Step 210) onto a point within the first subregion 162. The light focussed in the first subregion 162 is specularly reflected (Step 212) by the first reflective interface of the cornea 166 and some of the reflected first light returns (Step 214) to the second projection optics unit 160 via the port 170, whereupon the reflected first light propagates through the second projection optics unit 160 and is redirected by the beamsplitter 136 towards the first projection optics unit 156. Thereafter, the returning reflected light propagates through the first projection optics unit 156 and then propagates through the second lens 150 before entering the second fibre collimator 146 and propagating back (Step 214) to the second optical circulator 118. At the second optical circulator 118, the returning reflected first light is directed (Step 216) by the second optical circulator 118 to the confocal detector unit 148. The second optical circulator 118 serves to direct light received thereby to the third port 124 thereof and thus to the confocal detector unit 148.

The confocal detector unit 148 receiving the returning reflected first light generates (Step 218) a confocal detection signal. The confocal detection signal is analysed in a manner described later herein.

Turning to the low-coherence interferometric subsystem 102, the second light emitted by the SLED 106 is directed (Step 220) by the first optical circulator 110 to the second port 114 thereof before propagating to the optical splitter 126. The second light is then split (Step 220) by the optical splitter 126 and a first portion of the second light propagates (Step 222) to the focussing element 134 and a second portion of the second light propagates (Step 222) to the first fibre collimator 138, where the first and second portions of the second light are respectively collimated.

At the first fibre collimator 138, the first portion of the second light propagates through the first lens 144 and is focussed on to the reference mirror 142 before being retroreflected (Step 224) by the reference mirror 142. At the focussing element 134, the second portion of the second light propagates through the beamsplitter 136 (Step 226) and then propagates (Step 228) through the second projection optics unit 160 and is condition by the optical elements contained therein before exiting the second projection optics unit 160 and the apparatus 100 via the port 170 as collimated light that is incident upon the cornea 166 before being focussed (Step 230) by the cornea and the crystalline lens of the eye into the second subregion 164. The second light focussed into the second subregion 164 is reflected (Step 230) by the retina 164 and some of the reflected light returns to the second projection optics unit 160 via the port 170, whereupon the reflected light propagates through the second projection optics unit 160 and through the beamsplitter 136 (Step 228). Thereafter, the returning reflected light enters the focussing element 134 and propagates back to the splitter 126, whereupon the returning reflected light enters the splitter 126 and is directed (Step 232) to the first optical circulator 110. At the first optical circulator 110, the returning reflected light is directed (Step 234) by the first optical circulator 110 to the first port of the balanced detector unit 128. The first optical circulator 110 serves to direct light received thereby to the third port 116 thereof and thus the first port of the balanced detector unit 128.

Similarly, the retroreflected light from the reference mirror 142 propagates to the lens 144 and is collimated by the lens 144 prior to entering the first fibre collimator 138. Thereafter, the retroreflected light propagates to the splitter 126, whereupon the retroreflected light is directed (Step 236) by the splitter 126 towards the second port of the balanced detector unit 128 (Step 238).

The balanced detector unit 128 generates (Step 240) an interferometric signal in response to receipt of the retroreflected light originating from the reference mirror 142 and the reflected light of the first portion of the second light directed to the focussing element 134 in respect of the reference arm 132 and the measurement arm 130, respectively. Thereafter, the interferometric signal is analysed in a manner described later herein.

During irradiation of the sample 158 and generation of the confocal detection signal and the interferometric signal, the translatable carriage 140 is controlled by the translation controller 152 to scan (Step 242) the second lens 150 and the reference mirror 142 towards and away from the first projection optics unit 156 over a predetermined range of travel and at a predetermined frequency, thereby varying the interferometric signal and the confocal detection signal. In this regard, it should be appreciated that in this example a measurement cycle comprises a single translation or sweep in one direction of the second lens 150 and the reference mirror 142 while the second lens 150 and the reference mirror 142 are oscillating.

However, in other embodiments the measurement cycle can comprise more than one translation.

It should be appreciated that the low-coherence interferometric measurement system 102 images longitudinally in respect of the second subregion 164 and the confocal measurement subsystem 104 images longitudinally in respect of the first subregion 162. Furthermore, the longitudinal imaging by the interferometric measurement subsystem 102 and the confocal measurement subsystem 104 are substantially contemporaneous over the measurement cycle. In this regard, the second lens 150 constitutes a longitudinal imaging component. During translation of the translatable carriage 140, the translation controller 152 receives position information in respect of the translatable carriage 140 from the position encoder (not shown).

Turning to FIG. 3, the measurement unit 154 receives the confocal signal from the confocal detector unit 148 and the interferometric signal from the balanced detector 128. The measurement unit 154 cooperates with the translation controller 152 to receive a position feedback signal obtained by the translation controller 152 from the position encoder. The measurement unit 154 uses the position feedback signal in order to sample the confocal signal and the interferometric signal.

The measurement unit 154 uses (Step 300) the samples of the interferometric signal in order to detect (Step 302) a peak of an envelope in the time-varying interferometric signal as the translatable carriage 140 and hence the reference mirror 142 translates, the measure being in terms of pulses of the position feedback signal. When a peak is detected (Step 302), corresponding to a desired reflection from the retina 168, the measurement unit 154 records (Step 304) a count of the pulses received from the position encoder via the translation controller 152 from translation of the translatable carriage 140 until the peak was detected. The number of pulses that have been received in respect of the peak detected in the envelope of the interferometric signal constitutes an interferometric measurement, A, corresponding to an interferometrically measured range. Thereafter, the measurement unit 154 continues to measure translation of the translatable carriage 122 to detected peaks in samples of the interferometric signal (Steps 300 to 306) until such functionality is no longer required, for example when the apparatus 100 is powered down.

Likewise, the measurement unit 154 receives (Step 308) the samples of the confocal signal in order to detect (Step 310) peaks in the confocal signal, as the translatable carriage 140 and hence the second lens 150 translates, the measure again being in terms of pulses of the position feedback signal. When a peak is detected (Step 310), the measurement unit 154 records (Step 312) a count of the pulses being received from the position encoder via the translation controller 152 as the translatable carriage 140 translates. The count of the number of pulses that have been received in respect of the detected peak of the confocal signal constitutes a confocal measurement, B, corresponding to a confocal measured range. Thereafter, the measurement unit 154 continues to measure translation of the translatable carriage 140 between detected peaks in samples of the confocal signal (Steps 306 and 308 to 312) until such functionality is no longer required, for example when the apparatus 100 is powered down.

Using the ranges in respect of the interferometric measure, A, and the confocal measurement, B, the measurement unit 154 subtracts the confocal measurement, B, from the interferometric measurement, A, in order to obtain a difference measurement in terms of counts of pulses received from the position encoder. Using an assumed group refractive index for the sample 158, in this example, the eye, the distance between the cornea 166 and the retina 168, i.e. the axial distance, can be calculated by the measurement unit 154, taking into account an offset between a first point in the translation of the translatable carriage 140 corresponding to the scan of the first subregions by the confocal measurement subsystem 104 and a second point in the translation of the translatable carriage 140 corresponding to the scan of the second subregion by the low-coherence interferometric measurement subsystem 102. The calculated axial distance can then subsequently be output by an output device, for example a display. The group refractive index constitutes an effective refractive index, and in this example is determined for the media between and including the first and second subregions 162, 164 of the sample 158.

In this regard, the region to be measured comprises, in this example, a plurality of different contiguous subregions, including the first and second subregions 162, 164. The plurality of different contiguous subregions comprises a plurality of different refractive indices, respectively. As such, in other examples, the axial length between peaks in respect of the first and second subregions 162, 164 is calculated by calculating a plurality of distances between the first and second subregions 162, 164 using the plurality of different refractive indices, each refractive index respectively corresponding to a different subregion between the first and second subregions 162, 164. Interfaces between the contiguous subregions that reside between the first and second subregions give rise to corresponding peaks constituting reflections at the respective interfaces allowing the plurality of distances to be calculated. The plurality of distances calculated in respect of the different subregions is then summed to provide the distance between the first and second subregions 162, 164.

From the above examples, it should be appreciated that operation of the confocal measurement subsystem 104 comprising movement of the second lens 150 does not affect the interferometric measurement performed by the low-coherence interferometric measurement subsystem 102 comprising movement of the reference mirror 142. In this example, this is attributable to the fact that the scannable optical element is not disposed in the measurement arm 130 or the reference arm 132 of the low-coherence interferometric measurement subsystem 102 (or indeed the common sub-arm providing the internal optical path and formed in this example by the second projection optics unit 160). In this respect, the operation of the confocal measurement subsystem 104 does not constrain the optics of the low-coherence interferometric measurement subsystem 102 and confocal and interferometric measurements can be considered operationally independent of each other. For example, operation of the confocal measurement subsystem 104 does not influence a measurement result generated, when in use, by the interferometric measurement subsystem 102, i.e. the operation of one subsystem is substantially optically uninfluenced by the operation of the other subsystem. In this respect, translation of the second lens 150, for example, does not bias the interferometric measurement subsystem 102 so as to influence a measurement result generated by the interferometric measurement subsystem 102.

In any of the above examples, the optical measurement apparatus 100 can be calibrated to compensate for differences between measured distances to the first and/or second subregions 162, 164 in the sample 158. In this regard, it has been recognised that a first measurement of a distance to the retina 164 by the low-coherence interferometric measurement subsystem 102 differs in nature, for example scale, to a second substantially contemporaneous measurement of another distance to the cornea 166 by the confocal measurement subsystem 104. The difference between the two scales of measurement is attributable to the low-coherence interferometric measurement system 102 being linear, whereas the confocal measurement subsystem 104 possesses non-linearities. In order to be able to correct for such non-linearities in the confocal measurement subsystem 104, a dataset is compiled, in this example, to apply an offset to a distance measured by the confocal measurement system 104.

The dataset is compiled by providing a single-surface calibration target opposite the measurement port 170 of the optical measurement apparatus 100 and the calibration target is translated by predetermined increments over a range of positions corresponding to the scan range of the optical measurement apparatus 100. At each incremental position, the confocal subsystem 104 measure a distance to the single-surface target, the distance from the measurement port 170 being known. The difference between the known distance and the distance measured by the confocal measurement subsystem 104 is calculated and constitutes the offset. The offset value is stored in a lookup table with the distance measured by the confocal measurement subsystem 104.

The granularity of the dataset stored in the lookup table is initially dictated by the size of the predetermined increments of the position of the single-surface target. However, where greater resolution is required, interpolation between neighbouring values recorded in the lookup table can be calculated and stored in the lookup table.

Thereafter, during post-calibration normal operation of the optical measurement apparatus 100, a measurement made by the confocal measurement subsystem 104 can be looked up in the lookup table and corresponding offset values extracted and applied by the processing resource to the measurement made.

Although in the above example a lookup table is employed, a curve fitting technique can be employed by the processing resource to fit, for example, an analytical function to the offset values calculated. One suitable technique is a polynomial best fit technique. The fitted curve can then be referenced by the processing resource to determine an offset value for a corresponding confocal distance measurement.

The skilled person should appreciate that the above-described implementations are merely examples of the various implementations that are conceivable within the scope of the appended claims. However it should be appreciated that it is possible to use different wavelengths of electromagnetic radiation for the two measurement techniques employed by the apparatus 100.

It should also be appreciated that where at least partially coherent electromagnetic radiation is required for the confocal measurement subsystem 104, but where a common source of electromagnetic radiation is being employed, for example the SLED 106, the confocal measurement subsystem 104 can employ achromatic optics in order to ensure that no change in focal length with wavelength occurs and so the confocal point spread function is not broadened. In another embodiment, instead of achromatic optics, a narrowband filter can be employed to reduce the bandwidth of the light in this channel.

Although the above described examples employ a single reference arm 132, in other examples, a reference arm arrangement can be employed comprising a plurality of different and selectable optical path lengths. In such examples, a selection unit is employed and configured to select an optical path length from the plurality of different optical path lengths of the reference arm arrangement. The plurality of different optical path lengths can be achieved in a number of ways, for example by employing more than the single reflector element 142. A plurality of spaced reference optical reflector elements can be substantially coaxially disposed at different distances from the first fibre collimator 138. In such an example, the lens 144 can be carried by an independent translatable carriage separate from the translatable carriage 140 and the plurality of reference optical reflector element would not be carried by the translatable carriage 140. The selection unit can be operably coupled to the independent translatable carriage and configured to translate the lens 144 in response to a control signal generated by, for example, the measurement unit 154 so as to focus light emitted from the first fibre collimator 138 to a selected reference optical reflector element of the plurality of reference optical reflector elements. Each of the reference optical reflector elements can be partially reflective. In another example, the lens 144 can be mounted on the translatable carriage 140 and the fibre collimator 138 carried by the independent translatable carriage. In such an example, the single reference optical reflector element 142 can be employed, and can also be carried by the translatable carriage 140. The selection unit can be configured to control position of the independent translatable carriage and thus change an axial position of the first fibre collimator 138 disposed opposite the reference optical reflector element 142. In another example, instead of employing the plurality of reference optical reflector elements, a plurality of different selectable optical fibres can be employed. In this example, the single reference reflector 142 is employed and one of the plurality of different optical fibres is selected to emit light directed to the reference reflector 142. In this regard, each of the plurality of optical fibres is of a different length, each optical fibre being selectable by the selection unit. In another example, the plurality of different optical fibres can be of a common length and a plurality of different reference optical reflector elements can be disposed respectively at ends of the plurality of different optical fibres. In such an example, the optical distances of each reference optical reflector element of the plurality of difference reference optical reflector elements from the respective ends of the plurality of different optical fibres is different. In yet another example, the plurality of different selectable optical path lengths can comprise an optical fibre comprising a plurality of selectively reflective reflecting elements longitudinally spaced along the optical fibre. The plurality of selectively reflective reflecting elements can be operably coupled to the selection unit. In a further example, the plurality of different selectable optical path lengths can comprise an optical fibre and a plurality of reflecting elements, each of the plurality of reflecting elements being selectively disposable opposite an end of the optical fibre.

It should also be appreciated that the coupling of the low-coherence interferometric measurement subsystem 102 and the confocal measurement subsystem 104 to the beamsplitter 136 does not have to be in a particular order. In this regard, it should be understood that the low-coherence interferometric measurement subsystem 102 can be optically coupled to the second port of the beamsplitter 136 and the confocal measurement subsystem 104 can be optically coupled to the first port of the beamsplitter 136.

It should be appreciated that references herein to “light”, other than where expressly stated otherwise, are intended as references relating to the optical range of the electromagnetic spectrum, for example, between about 350 nm and about 2000 nm, such as between about 550 nm and about 1400 nm or between about 600 nm and about 1000 nm. The SLED 106 of the interferometric measurement subsystem can, for example, emit light in a near-infrared region of the electromagnetic spectrum. A wavelength of the light emitted by the SLED 106 can be less than 900 nm.

1. An optical measurement apparatus combining confocal measurement and low-coherence interferometric measurement, the apparatus comprising:
a housing;
a confocal measurement subsystem disposed within the housing;
an interferometric measurement subsystem disposed within the housing;
an optical combiner configured to provide the confocal measurement subsystem and the interferometric measurement subsystem with irradiative access to a region to be measured located at a substantially static target location external to the housing; and
a processing resource operably coupled to the confocal measurement subsystem and the interferometric measurement subsystem; wherein
the confocal measurement subsystem is configured to image longitudinally in respect of a first subregion in the region to be measured;
the interferometric measurement subsystem is configured to image longitudinally in respect of a second subregion in the region to be measured, the second subregion being axially spaced from the first subregion; and
the processing resource is configured to calculate a first range in respect of the first subregion and a second range in respect of the second subregion.

2. The apparatus according to claim 1, further comprising:
an optical path internal to the housing, the optical path extending from the optical combiner towards the region to be measured, the internal optical path being common to the confocal measurement subsystem and the interferometric measurement subsystem; and
a length of the internal optical path is fixed.

3. The apparatus according to claim 1, wherein
the processing resource is configured to calculate, when in use, a confocal measurement and an interferometric measurement substantially contemporaneously over a measurement cycle.

4. The apparatus according to claim 1, wherein the interferometric measurement subsystem is substantially optically uninfluenced, when in use, by operation of the confocal measurement.

5. The apparatus according to claim 1, wherein
the confocal measurement subsystem comprises at least one longitudinal imaging component;
the interferometric measurement subsystem comprises a measurement arm and a reference arm; and
neither the measurement arm nor the reference arm of the interferometric measurement subsystem comprises the at least one longitudinal imaging component.

6. The apparatus according to claim 2, wherein the length of the internal optical path is unchanged over the measurement cycle.

7. The apparatus according to claim 1, wherein
the confocal measurement subsystem is operationally independent of the interferometric measurement subsystem.

8. The apparatus according to claim 1, wherein the confocal measurement subsystem is operably coupled to the interferometric measurement subsystem.

9. The apparatus according to claim 1, further comprising:
projection optics disposed opposite a first side of the optical combiner; wherein
the interferometric measurement subsystem comprises a measurement arm configured to collimate, when in use, light propagating therethrough, an end of the measurement arm being disposed opposite a second side of the optical combiner.

10. The apparatus according to claim 1, wherein:
the interferometric measurement subsystem comprises:
a reference arm arrangement comprising a plurality of different selectable optical path lengths; and
a selection unit configured to select an optical path length of the plurality of selectable optical path lengths.

11. The apparatus according to claim 1, wherein the confocal measurement subsystem comprises a first translatable optical element and the interferometric measurement subsystem comprises a second translatable optical element, the first and second optical elements being configured to be translated substantially contemporaneously.

12. A method of measuring an axial length in a region to be measured located at a substantially static target location, the method comprising:
longitudinally imaging using a confocal measurement subsystem of an optical measurement apparatus in respect of a first subregion in the region to be measured in order to make a first measurement;
substantially contemporaneously longitudinally imaging using an interferometric measurement subsystem of the optical measurement apparatus in respect of a second subregion in the region to be measured in order to make a second measurement, the second subregion being axially spaced from the first subregion; and
calculating a first range in respect of the first subregion and a range in respect of the second subregion.

13. The method according to claim 12, further comprising:
providing a calibration target; and
calculating an offset between a first test measurement made by the confocal measurement subsystem in respect of the calibration target and a second test measurement made by the interferometric measurement subsystem in respect of the calibration target.

14. The method according to claim 12, further comprising:
determining an effective refractive index of the region to be measured; and
using the effective refractive index to make the first and second measurements.

15. The method according to claim 12, wherein the optical measurement apparatus emits a substantially collimated beam of light in respect of the interferometric measurement subsystem.
